(19) United States
(12) Patent Application Publication
(10) Pub. No.: US 2024/0321076 A1
(43) Pub. Date: Sep. 26, 2024

(54) DETECTION SYSTEM AND METHOD FOR EASY IMPLEMENTATION AT A FACILITY OR HOME

(71) Applicant: RSC ASSOCIATES, INC., Bowling Green, OH (US)

(72) Inventors: Matt Banach, Gurnee, IL (US); Joe Doran, Goliad, TX (US); Julia L. Caruso, Dublin, OH (US)

(21) Appl. No.: 18/599,904

(22) Filed: Mar. 8, 2024

Related U.S. Application Data
(60) Provisional application No. 63/491,972, filed on Mar. 24, 2023.

(51) Int. Cl. G08B 21/04

(52) U.S. Cl. CPC G08B 21/0461

(57) ABSTRACT

A detection system and method that is easy to implement and use for detecting wetness in an incontinence product is provided. The system may include a pod having a coupling for removably connecting the pod to the incontinence product, a sensor configured to detect wetness in the incontinence product, an optical label unique to the pod and assignable to the wearer, and a pod transmitter configured to transmit a first signal configured as a sub-gigahertz radiofrequency to a gateway. The gateway may be in cellular communication with a network and may transmit a second signal to a server. The server may transmit a third signal to a device used to monitor the wearer of the incontinence product. The present technology improves upon prior systems by providing an easy to implement wetness detection system utilizing long-range wireless transmission that does not require extensive infrastructure or complicated setup procedures.

### CROSS-REFERENCE TO RELATED APPLICATIONS

This application claims the benefit of U.S. Provisional Application No. 63/491,972, filed on Mar. 24, 2023. The entire disclosure of the above application is hereby incorporated herein by reference.

### FIELD

The present technology relates generally to incontinence products, and, more particularly, to systems and methods for using a detection system with an incontinence product.

### INTRODUCTION

This section provides background information related to the present disclosure which is not necessarily prior art.

Wetness indicators for incontinence products are well known in the art. Many existing products include visual indications disposed on the product to inform caregivers when the product requires changing due to wetness. For example, certain known absorbent articles have a wetness indicator strip that changes color upon sensing moisture. However, such visual indicators require caregivers to physically inspect the product, which may disrupt or bother the wearer.

More advanced detection systems utilize wireless transmission of wetness data to remote caregiver devices. However, these systems often have complex, multi-step setup procedures. For example, Bluetooth data transmission requires careful pairing procedures between detectors and monitoring devices. Additionally, Bluetooth covers only short transmission distances thereby requiring monitoring devices in every room or area of a facility. This extensive infrastructure increases costs and likelihood of interference from other wireless devices. Likewise, the use of Wi-Fi communication between detectors and gateways is problematic and typically requires access to facility IT infrastructure and Wi-Fi networks which may be difficult to implement and secure.

In addition to setup and infrastructure challenges, many known monitoring solutions require extensive battery charging maintenance. Others are susceptible to false alarms caused by sensor malfunctions or unintended radio interference. This can lead to inefficient caregivers as well as insufficient care of wearers. Several examples of known systems including wetness indicators are disclosed in U.S. Pat. Nos. 11,090,001, 11,147,490, U.S. Design Pat. No. D961,768, U.S. patent application Ser. Nos. 17/192,794, and 17/503,600, each of which is entirely incorporated herein by reference.

While many systems for monitoring incontinence products exist, they have a number of limitations such as difficult multi-step setup procedures, extensive infrastructure requirements, insufficient wireless transmission ranges, battery maintenance needs, sensor malfunctions, radio interference, and disruption of wearers or facility operations.

Accordingly, there is a continuing need for a system and method that detects wetness in an incontinence product that is easy to set up and use, can transmit data over longer distances, is reliable, and does not require a caregiver to be within a close proximity to the wearer to determine if the incontinence product is wet.

### SUMMARY

In concordance with the instant disclosure, a system and method that detects wetness in an incontinence product that is easy to set up and use, can transmit data over longer distances, is reliable, and does not require a caregiver to be within a close proximity to the wearer to determine if the incontinence product is wet, has surprisingly been discovered.

The present technology includes articles of manufacture, systems, and processes that relate to detecting wetness in an incontinence product using a long-range wireless transmission system that is easy to set up and implement.

In one embodiment, a detection system includes a pod having a coupling for removably connecting the pod to an incontinence product, a sensor configured to detect a wetness in the incontinence product, and a transmitter configured to transmit a first signal. The first signal is configured as a sub-gigahertz radiofrequency signal and includes a member selected from a group consisting of an indication of wetness in the incontinence product detected by the sensor, an identity of the pod, and combinations thereof.

The detection system further includes a gateway including a receiver configured to receive the first signal from the pod, and a transmitter in communication with a network and configured to transmit a second signal upon receipt of the first signal. The second signal includes a member selected from a group consisting of the indication of wetness in the incontinence product detected by the sensor, the identity of the pod, the identity of the gateway, and combinations thereof.

A server including a receiver may be configured to be in communication with the network and configured to receive the second signal from the gateway, and a transmitter configured to transmit a third signal using the network upon receipt of the second signal. The third signal includes a member selected from a group consisting of the indication of wetness in the incontinence product detected by the sensor, the identity of the pod, the identity of the gateway, and combinations thereof.

The detection system includes a monitoring device including a receiver configured to be in communication with the network and configured to receive the third signal from the server, and a notification means configured to provide a notification based upon the third signal. The notification includes a member selected from a group consisting of the indication of wetness in the incontinence product detected by the sensor, the identity of the pod, and combinations thereof.

In a particular embodiment, a detection system includes a pod with a coupling for removably connecting the pod to an incontinence product. The pod includes a sensor to detect wetness in the incontinence product and a transmitter to transmit a first signal. The first signal is a sub-gigahertz radiofrequency signal that includes an indication of detected wetness and/or an identity of the pod.

The system also includes a gateway with a receiver to receive the first signal from the pod. The gateway has a transmitter to communicate with a network and transmit a second signal upon receiving the first signal. The second signal includes the detected wetness indication, pod identity, or both.

The system further includes a server with a receiver to communicate with the network and receive the second signal. The server has a transmitter to transmit a third signal on the network upon receiving the second signal. The third signal includes the detected wetness indication, pod identity, or both.

Additionally, the system includes a monitoring device with a receiver to communicate with the network and receive the third signal. The monitoring device provides notifications based on the third signal, including the detected wetness indication, pod identity, or both.

The first and second signals may use a custom electronic data interchange protocol. The gateway communicates with the network using a cellular connection, and the server may communicate with the network using a cellular connection. The pod batteries may be rechargeable and last at least one year before replacement, and the pod may include battery-conserving firmware enabling a sleep mode during which the pod consumes no battery. The pod may automatically connect to the gateway upon powering on and/or when transmitting the first signal. The gateway may include one or more antennas, backup batteries, SD Cards for data storage, displays, indicator lights, and/or power cords.

The gateway and pod may have unique serial numbers for identification, remote identification, remote access, and remote or onsite management. Updates and configurations may be handled remotely via the server. The radiofrequency signal may transmit at 915 MHz to minimize interference. Pod hardware may include a sensor, battery, circuit board, case, and coupling. The monitoring device may include an installable software application displaying a dashboard with statuses of multiple individuals and customizable alerts and notifications.

The pod sensor may utilize conductive ink on the incontinence product to detect wetness, and the pod may include an optical label with a QR code. The pod may enter sleep mode during non-use to conserve battery and have a start function to awaken from sleep. The server may store data for automatically configuring new gateways and pods, and the gateway may connect to a retained power cord for continuous power.

Pod communication may be initiated by engaging the pod's start function, and the pod may auto-configure using gateway data. The pod identity is assigned to a wearer using the unique QR code, and the pod is coupled to an incontinence product. The sensor detects wetness triggering the first signal to the gateway, the second signal to the server, and third signal to the monitoring device for monitoring wetness. The first and second signals may use a custom protocol. The gateway may communicate with the network via cellular communication, and engaging by the start functions of the pod and/or the gateway may initiate communication. The gateway and pod may auto-configure by loading data settings from the server and gateway. Configurations and firmware updates may be remotely managed via the server. A sleep mode may conserve pod battery during non-use. The monitoring means may include a mobile device application. Statuses of multiple individuals may be displayed, and alerts and notifications may be configurable.

In another embodiment, a method of using a detection system includes steps of providing a pod having a coupling for removably connecting the pod to an incontinence product, a sensor configured to detect a wetness in the incontinence product, an optical label unique to the pod and assignable to the wearer, and a transmitter configured to transmit a first signal. The first signal is configured as a sub-gigahertz radiofrequency signal and includes a member selected from a group consisting of an indication of wetness in the incontinence product detected by the sensor, an identity of the pod, and combinations thereof.

The method further includes providing a gateway including a receiver configured to receive the first signal from the pod and a transmitter in communication with a network and configured to transmit a second signal upon receipt of the first signal. The second signal includes a member selected from a group consisting of the indication of wetness in the incontinence product detected by the sensor, the identity of the pod, identity of the gateway and combinations thereof.

A further step of providing a server including a receiver configured to be in communication with the network and configured to receive the second signal from the gateway and a transmitter configured to transmit a third signal using the network upon receipt of the second signal is included. The third signal includes a member selected from a group consisting of the indication of wetness in the incontinence product detected by the sensor, the identity of the pod, identity of the gateway and combinations thereof.

The method further includes providing a monitoring device including a receiver configured to be in communication with the network and configured to receive the third signal from the server and a notification means configured to provide a notification based upon the third signal. The notification includes a member selected from a group consisting of the indication of wetness in the incontinence product detected by the sensor, the identity of the pod, and combinations thereof.

Additional steps may be included such as installing or placing the gateway at a desired location, initiating communication between the transmitter of the gateway and the network by at least one of connecting the gateway to a power source and engaging a start function on the gateway, auto-configuring or configuring the gateway using a first set of data from the server, initiating the transmitter of the pod and the first signal by engaging a start function on the pod, and auto-configuring or configuring the pod using a second set of data from the gateway.

Additional steps may include assigning the identity of the pod to the wearer using a unique optical label displayed on the pod, coupling the pod to the incontinence product, detecting the wetness in the incontinence product using the sensor, sending the sub-gigahertz radiofrequency signal from the pod to the gateway, sending the second signal from the gateway to the server, sending the third signal from the server to the monitoring device, and monitoring the wetness in the incontinence product using the notification means. The method may further include a step of downloading a monitoring application that communicates with the server and entering wearer identification information.

In another embodiment, the method of using the system may include providing the detection pod, gateway, server, and monitoring device, installing the gateway at a location and initiating communication by powering on and/or engaging the gateway's start function. The gateway may auto-configure using server data.

In an exemplary embodiment, a detection system and method for monitoring wetness in incontinence products by caregivers is designed for easy at-home or facility implementation without complex setup requirements. Key features include a wireless pod with a removable coupling to attach to an incontinence product, a sensor to detect wetness, and a transmitter capable of sending sub-gigahertz radio signals to a gateway regarding detected wetness and/or pod identity. The gateway automatically receives and forwards signals via a cellular network to a server, which conveys information to a monitoring application.

The system uniquely leverages long-range wireless protocols, custom interchange signaling such as a custom electronic data interchange protocol, cellular connectivity, remote access capabilities, optimized power consumption, and streamlined automatic configuration to enable effortless use for residents and caregivers without reliance on facility infrastructure. In particular, the long-range wireless performance provides complete coverage to avoid close physical checks while conserving battery through a sleep mode, and the custom protocol prevents interference with other devices while eliminating difficult manual pairing, set-up, and management. Simplified monitoring, alerts, and data reporting are facilitated through the monitoring application.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a block diagram illustrating a detection system, according to some embodiments of the present disclosure.

FIG. 2 is a flowchart illustrating a detection system, according to certain embodiments of the present disclosure.

FIG. 3 is a drawing of a pod, according to certain embodiments of the present disclosure.

FIG. 4A is a flowchart illustrating a method of using a detection system, according to certain embodiments of the present disclosure.

FIG. 4B is a flowchart extending from FIG. 4A and further illustrating the method of using a detection system, according to certain embodiments of the present disclosure.

FIG. 4C is a flowchart extending from FIG. 4B and further illustrating the method of using a detection system from FIG. 4A, according to certain embodiments of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves upon prior systems by providing an easy to implement wetness detection system utilizing long-range wireless transmission that does not require extensive infrastructure or complicated setup procedures.

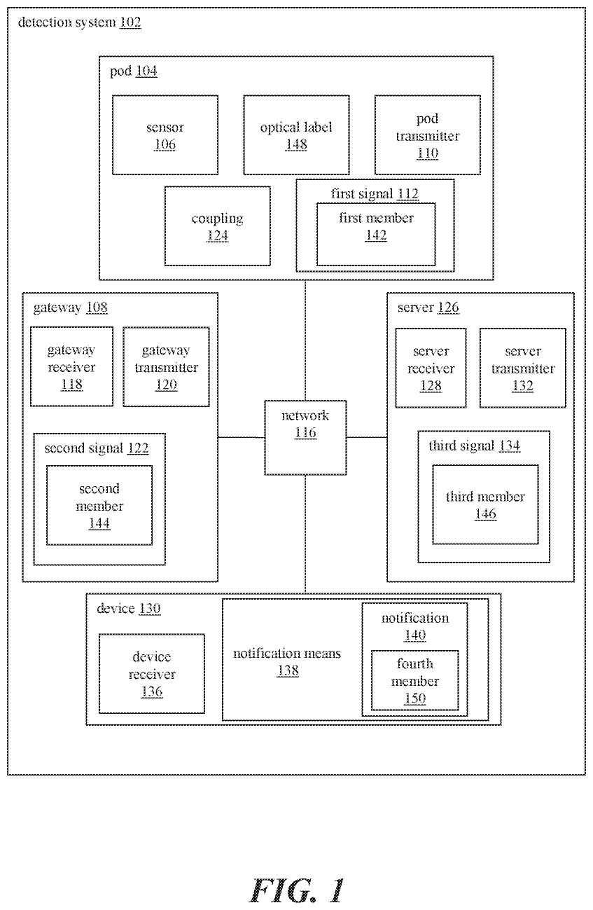

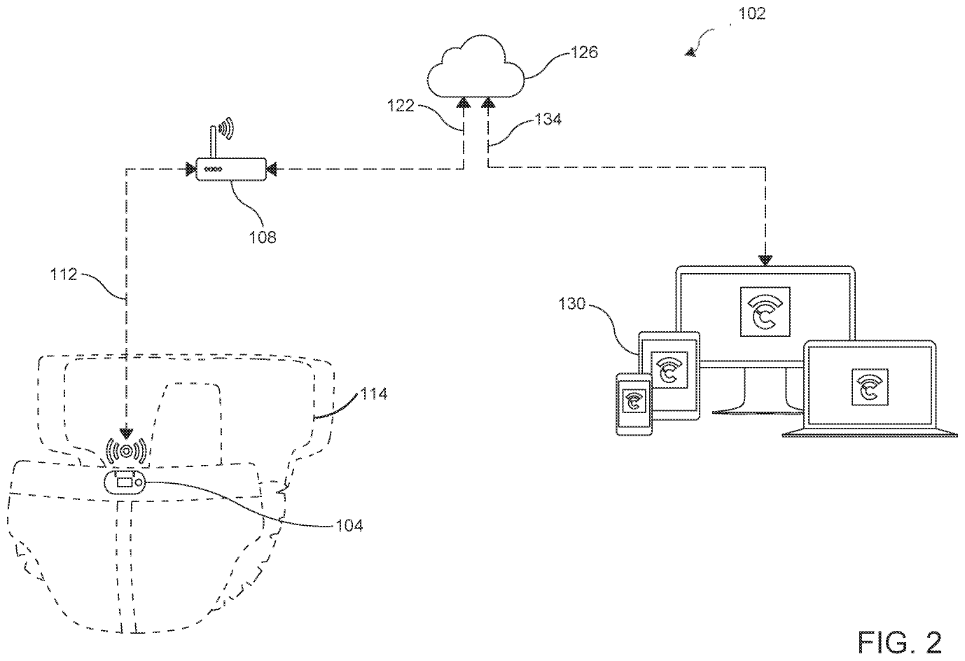

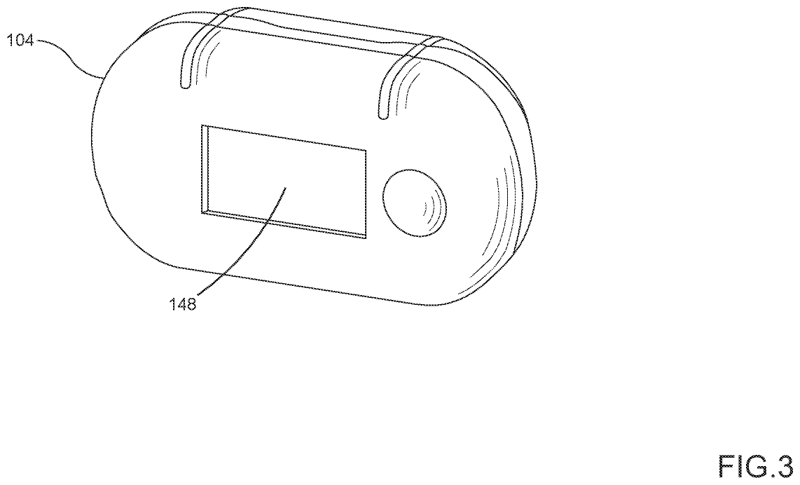

In certain embodiments, as shown in FIGS. 1-3, a detection system 102 includes a pod 104. The pod 104 includes a sensor 106 for detecting wetness in an incontinence product 114 and a pod transmitter 110 configured to transmit a first signal 112. The pod 104 may further include a coupling 124 for removably connecting the pod 104 to the incontinence product 114. In certain embodiments, the pod 104 may include a receiver.

The detection system 102 further includes a gateway 108 having a gateway receiver 118 configured to receive the first signal 112 from the pod 104. The gateway receiver may be configured to receive instructions and remote updates, according to certain embodiments. The gateway 108 also includes a gateway transmitter 120. The gateway transmitter 120 communicates with a network 116 and is configured to transmit a second signal 122 upon receiving the first signal 112. The gateway transmitter 120 may also be configured to communicate with a receiver of a pod 104.

The detection system 102 additionally includes a server 126 having a server receiver 128 configured to communicate with the network 116 and receive the second signal 122 from the gateway 108. The server 126 includes a server transmitter 132. The server transmitter 132 is configured to transmit a third signal 134 using the network 116 upon receiving the second signal 122 from the gateway 108. In certain embodiments, the server may store initialization data for automatically configuring the gateway 108 and the pod 104 when one or both of the pod 104 and the gateway 108 are powered on.

A device 130 such as a monitoring device may be included with the detection system 102, according to certain embodiments. The device 130 has a device receiver 136 configured to communicate with the network 116 and receive the third signal 134 from the server 126. The device 130 may include a notification means 138 for providing a notification 140 regarding the incontinence product 114, such as an indication of wetness, to a caregiver.

In certain embodiments, this first signal 112 may include a first member 142 selected from a group including at least one of an indication of wetness detected by the sensor 106, an identity of the pod 104, or both. The first signal 112 may be a sub-gigahertz radiofrequency signal, according to certain embodiments. Upon receipt of the first signal 112 from the pod 104 by the gateway 108, the second signal 122 from the gateway 108 may include a second member 144 selected from a group including at least one of an indication of wetness detected by the sensor 106, the identity of the pod 104, the identity of the gateway 108, or combinations thereof. Upon receipt of the second signal 122 from the server 126, the third signal 134 from the server 126 may include a third member 146 selected from a group including at least one of an indication of wetness detected by the sensor 106, the identity of the pod 104, the identity of the gateway 108, or combinations thereof. The notification 140 from the notification means 138 may include a fourth member 150 selected from a group including at least one of an indication of wetness detected by the sensor 106, the identity of the pod 104, or both.

In certain more particular embodiments, at least the first signal 112 is transmitted using a custom electronic data interchange protocol, and at least the gateway 108 and the network 116 may communicate using a cellular connection. As used herein, the term “custom electronic data interchange protocol” is defined as being a proprietary or customized set of rules, formats, and standards for electronically transferring data between devices. Unlike standard electronic data interchange protocols, the custom electronic data interchange protocol of the present disclosure is specifically designed to exchange data in a way that only components of the detection system 102 will recognize.

The second signal 122 may be transmitted using the same custom electronic data interchange protocol, a different custom electronic data interchange protocol, or any suitable means of transmission. The server 126 may also communicate with the network 116 using any suitable means of transmission and the device may communicate with the network using a cellular connection, Wi-Fi connection or any suitable means of transmission. Alternative means of communication may be used according to certain embodiments. Batteries (not shown) for the pod 104 may be rechargeable, and the pod 104 may operate for at least one year before requiring battery replacement. Additionally, the pod 104 may include battery-conserving firmware configured to enable a sleep mode during which little to no battery is consumed. The battery-conserving firmware may also limit power to just the sensor 106 until a significant change in wetness is detected before enabling other pod 104 functionality which may include sending of the first signal 112.

In certain embodiments, the pod 104 may automatically connect to the gateway 108 upon initiating transmission of the first signal 112. The pod 104 may include an optical label 148 displaying a QR code or other unique identifier and enter a battery-conserving sleep mode during non-use. The pod 104 may be awakened from sleep mode using a start function.

The pod 104 may include additional hardware components (not shown) such as a circuit board, a case, and a coupling for removably connecting the pod 104 to the incontinence product 114, as non-limiting examples, and the gateway 108 may include one or more of an antenna, display, indicator light, power cord to connect to a power source, and any other suitable components, as non-limiting examples, in certain embodiments. The gateway 108 and the pod 104 may each include a unique serial number for purposes of identification, remote access, and/or any other desirable function. Updates and configurations for the gateway 108 and/or the pod 104 may be operated remotely via the server 126 or onsite using one or more of the pod 104, the gateway 108, and the device 130. In one more particular embodiment, at least the first signal 112 may be a radiofrequency signal that transmits at 915 MHz to minimize interference with other wireless devices. In certain embodiments, at least the first signal 112 may be a radiofrequency signal that transmits at 433 MHz to minimize interference with other wireless devices. A skilled artisan may use any suitable radiofrequency to minimize interference, as desired.

In certain embodiments, the detection system 102 includes the pod 104, the gateway 108, the server 126, and the device 130. The pod 104, may be removably coupled to the incontinence product 114, may include the sensor 106 configured to detect wetness in the incontinence product 114, and may include the optical label 148 that is unique to the pod 104 and assignable to the wearer. In certain embodiments, the optical label 148 may include a quick response code (QR code).

The pod 104 may also include the pod transmitter 110 configured to transmit the first signal 112, and the first signal 112 may be configured as a sub-gigahertz radiofrequency signal. The first signal 112 may include the first member 142 selected from a group including at least one of the indication of wetness in the incontinence product 114 detected by the sensor 106, the identity of the pod 104, and combinations thereof. In certain embodiments, the pod 104 may transmit additional data sets including information such as a battery level of the pod 104, a temperature of the wearer, a saturation level of the incontinence product 114, and any other useful information relating to the pod 104, the wearer, the incontinence product 114, and the detection system 102, as non-limiting examples. Each of the first member 142, the second member 144, the third member 146, and the fourth member 150 may be selected from any data set or group, as determined by one of skill in the art.

The pod 104 may be designed to automatically connect to the gateway 108 using LoRa communication once the pod 104 is powered on. The pod 104 may be powered on using a button or other start function or may automatically engage with the gateway 108 when the gateway 108 is powered on. The pod 104 may communicate with more than one gateway 108, as needed, and may jump from one gateway 108 to another with respect to communication, as needed.

In certain embodiments, the gateway 108 may include the gateway receiver 118 configured to receive the first signal 112 from the pod 104 and the gateway transmitter 120 in communication with the network 116 and configured to transmit the second signal 122 upon receipt of the first signal 112. The second signal 122 may include the second member 144 selected from a group including at least the indication of wetness in the incontinence product 114 detected by the sensor 106, the identity of the pod 104, the identity of the gateway 108, battery level of the pod 104, a temperature of the wearer, and any other useful information relating to the pod 104, the wearer, the incontinence product 114, and the detection system 102, as non-limiting examples. The gateway 108, according to certain embodiments, may be powered on by one or more of plugging in the gateway 108 and/or engaging a start function. The gateway 108 may include a visual display in certain embodiments. Advantageously, the gateway 108 is able to communicate with a large number of pods 104 over longer distances using LoRa communications. The gateway 108 is always engaged and available to receive communications from each pod 104, according to certain embodiments.

The server 126 may include the server receiver 128 configured to be in communication with the network 116 and configured to receive the second signal 122 from the gateway 108 and server transmitter 132 configured to transmit the third signal 134 using the network 116 upon receipt of the second signal 122. The third signal 134 may include the third member 146 selected from a group including at least the indication of wetness in the incontinence product 114 detected by the sensor 106, the identity of the pod 104, the identity of the gateway 108, battery level of the pod 104, a temperature of the wearer, and any other useful information relating to the pod 104, the wearer, the incontinence product 114, and the detection system 102, as non-limiting examples. The server 126 may communicate with other components of the detection system 102 using various communication and network types, including the internet, cellular communication, Wi-Fi, local area network (LAN) communication, individual cellular plans, cell towers, or any other suitable means for communicating within and throughout the detection system 102.

In certain embodiments, the device 130 may include a device receiver 136 configured to be in communication with the network 116 and configured to receive the third signal 134 from the server 126. In certain embodiments, the device 130 may include a software application (not shown) configured to receive the third signal 134 from the server 126. The device 130 may include the notification means 138 configured to provide the notification 140 based upon the third signal 134. The notification 140 may include at least the indication of wetness in the incontinence product 114 detected by the sensor 106, the identity of the pod 104, and combinations thereof. In certain embodiments, the software application may be downloaded and utilized using the device 130 to evaluate, manipulate, and display data provided by the first, second, and third signals 112, 122, 134, and any other suitable means for collecting data, such as user input, in one non-limiting example. Data may be displayed using the notification means 138. Non-limiting examples of data sets that may be collected and provided using the first, second, and third signals 112, 122, 134, or any other suitable means, include identifying information relating to the wearer and/or the pod 104, historical information relating to one or both of the wearer and the pod 104, time elapsed, indications of wetness, percent of saturation, temperature of wearer, and any other desired data. The device 130 may communicate with other components of the detection system 102 using various communication and network types, including the internet, cellular communication, Wi-Fi, local area network (LAN) communication, individual cellular plans, cell towers, or any other suitable means for communicating within and throughout the detection system 102.

Advantageously, the detection system 102 utilizes communication means designed to prevent interoperating with other technology, such as cellular communication and custom electronic data interchange protocols, as non-limiting examples. In certain embodiments, the gateway 108 and the pod 104 each have a serial number programmed into a computer chip that provides the gateway 108 and the pod 104 with a unique identification allowing for the custom protocol to automatically start-up, configure and communicate. As a result, no time is required to manually register and configure the pod 104 and gateway 108, making set up faster and easier.

Additionally, according to certain embodiments, the optical label 148 allows the pod 104 to be assigned to an individual wearer using the software application available on the device 130. Assignment of the pod 104 may be an easy 1-step process. The pod 104 firmware may be designed to conserve its battery prior to initiating use of the pod 104 and any time the pod is not assigned to a wearer and/or disposed on an incontinence product 114. As a result, no time is required for installing batteries prior to the initial use of the pod 104, making set up faster and easier. The pod 104 may be stored in a sleep mode during which no battery consumption is required.

In certain embodiments, the pod 104 has a start function that when engaged “wakes up” the pod 104. If the pod 104 is not assigned to a wearer, the pod 104 will go back to sleep. After assignment to a wearer, the pod 104 may not continuously send the first signal 112; rather the pod 104 may send the first signal 112 when the sensor 106 detects a change in wetness in the incontinence product 114. As a result, the pod 104 continues to conserve its battery resulting in easy system 102 usage as the battery does not need to be changed or recharged every day or every few weeks. In certain embodiments, batteries used with the pod 104 may be rechargeable.

Advantageously, according to certain embodiments, the gateway 108 is designed to be a passthrough for the first signal 112 received from the pod 104 and sent directly to the server 126 with no setup required. The pod 104 and the gateway 108 may be aligned to the same custom and proprietary communication protocol for easy set up and transfer of information. According to certain embodiments, the pod 104 may automatically connect to the gateway 108. The gateway 108 is always on and listening for the custom communication protocol or custom electronic data interchange protocol, in certain embodiments. Once the gateway 108 receives the first signal 112 from the pod 104, the gateway 108 automatically passes the second signal 122 using cellular communication to the server 126, and the server 126 passes the third signal 134 to the device 130 including the software application.

In certain embodiments, if more than one gateway 108 receives the first signal 112, the detection system 102 will process the first signal 112 from one gateway 108 and use transmission time to determine if there are multiple gateways 108 sending or receiving the same first signal 112. Gateway 108 firmware updates may be automatically distributed using any suitable type of communication. The pod 104 configuration including settings for usage as well as updates may be automatically received by way of the gateway 108. In certain more particular embodiment, a 915 MHz frequency may be used to advantageously minimize interference with other devices such as personal devices and healthcare devices, as non-limiting examples.

Advantageously, the detection system 102 is easy to set up and easy to use. During set up, configuration of hardware is simple and automatic, no pairing is required, no registration is required, communication between hardware components is longer range, installation of hardware is minimal, and batteries do not need to be charged or installed. Set up does not require on-site implementation support or help from IT professionals.

Further advantageously, the detection system 102 uses long-range (LoRa) communications in conjunction with a proprietary communications protocol such as custom electronic data interchange protocols, in certain embodiments. This allows a single gateway 108 to service a large area within a facility or home and individual gateways 108 are not needed in each room. LoRa is a radio communication technique that uses sub-gigahertz radio frequency bands worldwide. LoRa enables long-range transmissions with low power consumption. Advantageously, LoRa is always present and always transmitting over longer ranges. The detection system 102 also uses other forms of communication, such as cellular communication as one non-limiting example, and is designed to work out-of-the-box at any location that has cellular coverage or other means for communication. The detection system 102 begins working immediately when the gateway 108 is powered on. Wi-Fi is not required for set up or use of the detection system 102. In certain embodiments, the detection system 102 uses LoRa such that communication occurs in a closed loop, thereby militating against interference with other devices not included in the detection system 102.

Communication between any combination of the pod 104, the gateway 108, the network 116, the server 126, and the device 130 may only be recognized by the detection system 102, according to certain embodiments. In one particular embodiment, at least the first signal 112 may be sent from the pod 104 to the gateway 108 using a custom or unique electronic data interchange protocol recognized only by the detection system 102. In certain embodiments, at least the first signal 112 may be sent from the pod 104 to the gateway 108 using LoRa radio communication. Advantageously, the custom electronic data interchange protocol permits only the pod 104 and the gateway 108 to communicate with respect to data being transmitted. LoRa radio communication may be used to transmit the data, however LoRa radio communication is simply transmitting the data and is not otherwise in communication with the pod 104 and/or the gateway 108. It should be appreciated that, if a communication is sent by one or both of a pod 104 or a gateway 108 and the communication does not have an assigned facility, the communication may be automatically assigned to the facility in which the wearer is registered or assigned. As such, one or both of the pod 104 and the gateway 108 may have a “self-registration” component included in the detection system 102.

In certain embodiments, the custom electronic data interchange protocol may utilize a unique key such as a serial number, as one non-limiting example, recognized only by any combination of the pod 104, the gateway 108, the network 116, the server 126, and the device 130 of the detection system 102. Any suitable means for transmitting data that can only be recognized by any combination of the pod 104, the gateway 108, the network 116, the server 126, and the device 130 of the detection system 102 may be employed, as determined by one of skill in the art.

In certain embodiments, any one or more combinations of the gateway 108, the network 116, the server 126, and the device 130 may be in cellular communication with one another using any suitable means such as a cellular chip and/or a cellular plan or device platform for IOTs, as non-limiting examples.

Data transmitted using one or more of the first signal 112, the second signal 122, the third signal 134, or any other suitable means, may be encrypted by transforming the data into a code that can only be read by the detection system 102. Encryption may be symmetric, asymmetric, hybrid, or any combination thereof and may employ different algorithms and keys for encrypting and decrypting the data. Data may be transmitted using secure protocols, and access to data and resources may be controlled using predefined criteria, such as roles, permissions, and policies, according to certain embodiments. Any suitable means for securing and protecting the data transmitted using the detection system 102 may be utilized, as determined by one of skill in the art.

Advantageously, at least the pod 104 and the gateway 108 communicate in a unique way, such as by organizing data as a unique data set only readable and/or recognized by the pod 104 and the gateway 108, in one non-limiting example, and the unique transfer of data is only recognized within the detection system 102. Any combination of the pod 104, the gateway 108, the network 116, the server 126, and the device 130 may be configured to receive, transmit, read, and/or recognize the unique data transmitted using any one or more of first signal 112, the second signal 122, the third signal 134, or any other suitable means.

The detection system 102 uniquely leverages long-range wireless protocols, custom interchange signaling such as a custom electronic data interchange protocol, in one non-limiting example, cellular connectivity, remote access capabilities, optimized power consumption, and streamlined automatic configuration to enable effortless set-up and use of the detection system 102. In particular, the long-range wireless performance provides complete coverage without relying on Wi-Fi, Bluetooth, multiple gateways, and wired internet. Additionally, data travels longer distance without the need for multiple gateways, such as in a mesh network setup. The custom electronic data interchange protocol eliminates security issues and interference with outside devices. The setting up of the detection system 102 is automatic and does not require extensive time and resources.

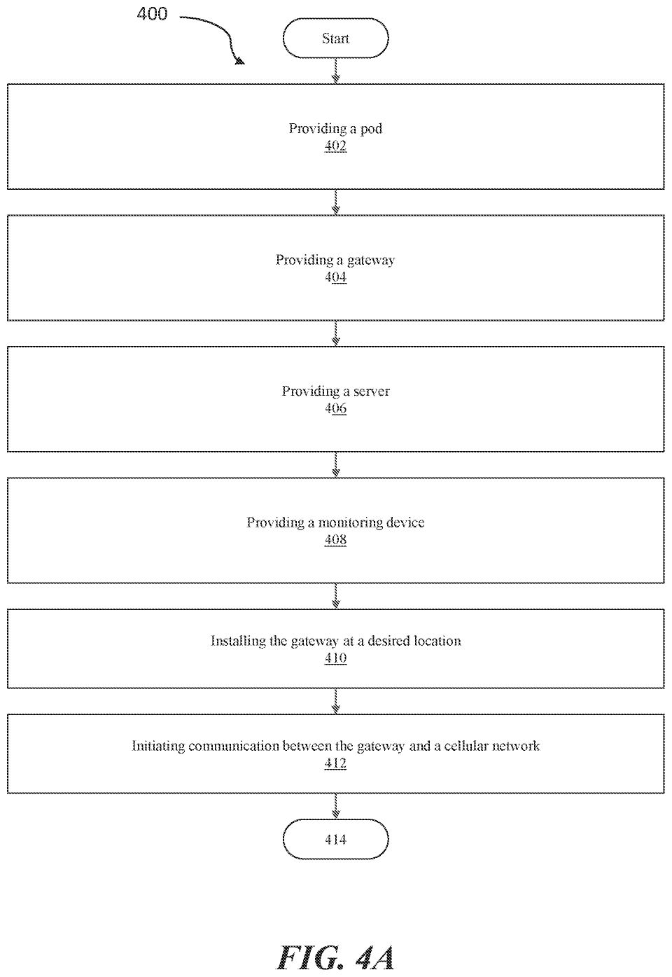

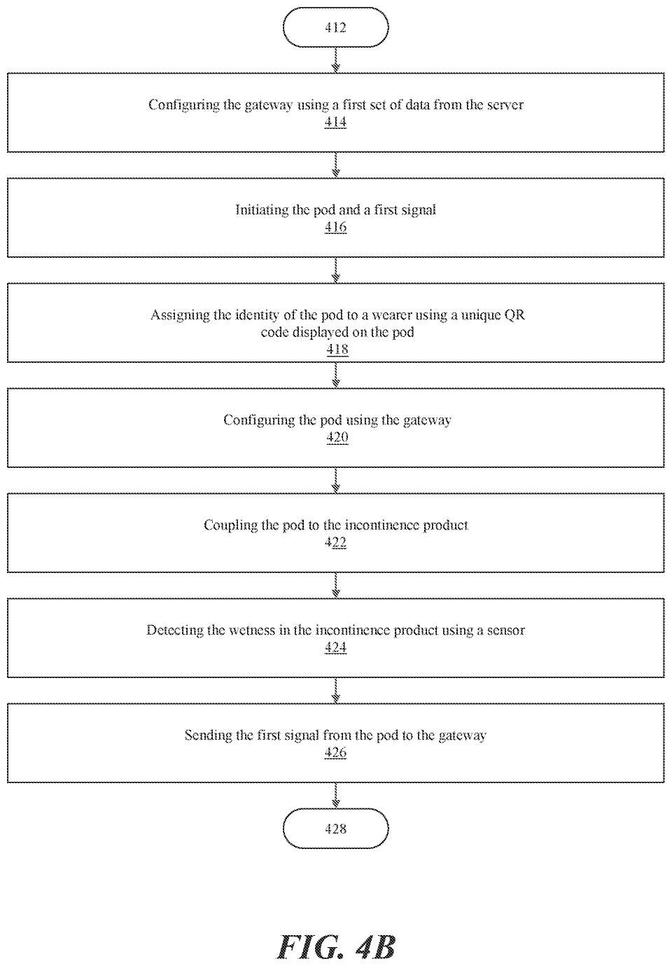

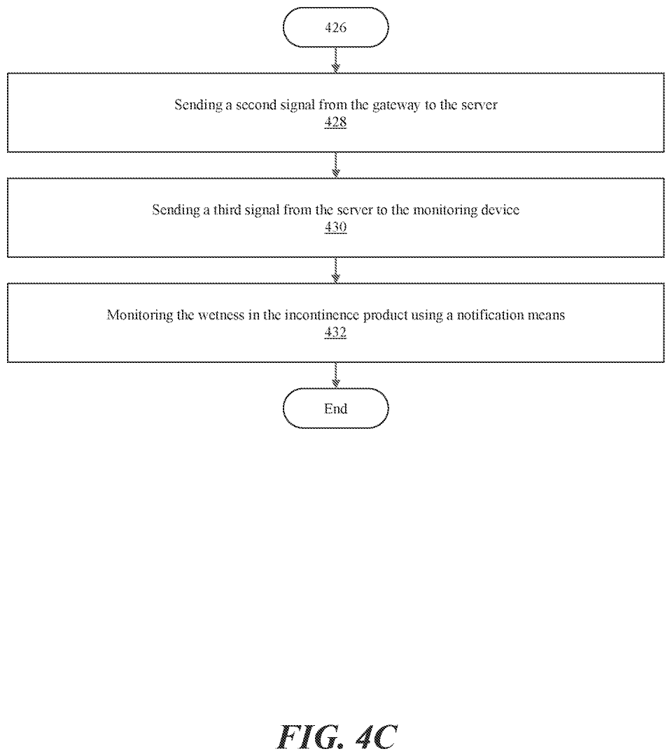

FIGS. 4A-4C include flowcharts describing a method of using the detection system 102, according to certain embodiments of the present disclosure. The method may include the step 402 of providing a pod 104. The pod 104 may be removably coupled to an incontinence product 114 and may include a sensor 106 configured to detect wetness in the incontinence product 114 and an optical label 148 unique to the pod 104 and assignable to the wearer. The pod 104 may also include a pod transmitter 110 configured to transmit a first signal 112, and the first signal 112 may be configured as a sub-gigahertz radiofrequency signal. The first signal 112 may include a first member 142 selected from a group including an indication of wetness in the incontinence product detected by the sensor 106, an identity of the pod 104, and combinations thereof.

According to certain embodiments, the method may include the step 404 of providing a gateway 108. The gateway 108 may include a gateway receiver 118 configured to receive the first signal 112 from the pod 104 and a gateway transmitter 120 in communication with a network 116 and configured to transmit a second signal 122 upon receipt of the first signal 112. The second signal 122 may include a second member 144 selected from a group including an indication of wetness in the incontinence product detected by the sensor 106, an identity of the pod 104, an identity of the gateway 108, and combinations thereof.

In certain embodiments, the method may include the step 406 of providing a server 126. The server 126 may include a server receiver 128 configured to be in communication with the network 116 and configured to receive the second signal 122 from the gateway 108 and a server transmitter 132 configured to transmit a third signal 134 using the network 116 upon receipt of the second signal 122. The third signal 134 may include a third member 146 selected from a group including an indication of wetness in the incontinence product detected by the sensor 106, the identity of the pod 104, the identity of the gateway 108, and combinations thereof. It should be appreciated that a single server may be provided for many detection systems 102 within scope of present disclosure.

In certain embodiments, the method may include the step 408 of providing a device 130. The device 130 may include a device receiver 136 configured to be communication with the network 116 and configured to receive the third signal 134 from the server 126. The device 130 may include a notification means 138 configured to provide a notification 140 based upon the third signal 134. The notification 140 may include a fourth member 150 selected from a group including an indication of wetness in the incontinence product detected by the sensor 106, the identity of the pod 104, and combinations thereof. It should be appreciated that one or more devices 130 may be provided by a facility and may not be provided with the components of the detection system 102.

In certain embodiments, the method may include additional steps. As non-limiting examples, a step 410 may include installing or placing the gateway 108 at a desired location, and a step 412 may include initiating communication between the gateway transmitter 120 and the network 116 by at least one of connecting the gateway 108 to a power source and engaging a start function on the gateway 108. Further steps may include a step 414 of auto-configuring the gateway 108 using a first set of data from the server 126, and a step 416 may include initiating the pod transmitter 110 and the sub-gigahertz radiofrequency first signal 112 by engaging a start function on the pod 104.

According to certain embodiments, a step 418 may include assigning the identity of the pod 104 to the wearer using a unique QR code displayed on the pod 104, a step 420 may include auto-configuring the pod 104 using the gateway 108, a step 422 may include coupling the pod 104 with the incontinence product 114, and a step 424 may include detecting the wetness in the incontinence product using the sensor 106. Additional steps may include a step 426 of sending the first signal 112 from the pod 104 to the gateway 108, a step 428 of sending the second signal 122 from the gateway 108 to the server 126, a step 430 of sending the third signal 134 from the server 126 to the device 130, and a step 432 of monitoring the wetness in the incontinence product 114 using the notifications 140 provided by the notification means 138. Additional steps may include downloading a software application on the device 130, the software application in communication with the server 126, and entering information identifying a wearer of the incontinence product 114 using the software application (not shown).

In certain embodiments, the method 400 may include placing a gateway 108 in a desired location, plugging in the gateway 108, auto-configuring the gateway 108 using the network 116, turning on the pod 104, and auto-configuring the pod 104 using the gateway 108. In certain embodiments the method 400 may include remotely managing the gateway 108 and the pod 104 using one or more unique serial numbers. One or more steps included in the method 400 may be repeated, omitted, or performed in any desirable alternative order, as needed. Any suitable steps necessary for setting up and using the detection system 102 may be included in the method 400, as determined by someone skilled in the art.

### EXAMPLES

Example embodiments of the present technology are provided with reference to the several figures enclosed herewith.

Below in TABLE 1 is a comparison of key features for the system and method of the present disclosure relative to prior products on the market:

TABLE 1

| Feature | System and Method | Prior Products |
|---|---|---|
| Hardware Setup | No manual configuration, pairing or | Complex, costly setup, IT |
| Requirements | registration | support often needed |
| Wireless Protocol | Long-range LoRa + Cellular | Short-range Bluetooth + |
| | | Wi-Fi |
| Infrastructure | Works anywhere with cellular signal | Requires facility Wi-Fi |
| Needs | | access |
| Battery Life | 1+ years, sleep mode optimization | Frequent charging required |
| | | or changing of coin batteries |
| Wearer detection | Entire facility coverage with LoRa | Room coverage with |
| Range | | Bluetooth |
| Number of | Typically 1-3 per facility | One per room or several |
| gateways | | rooms |
| Interference | Closed LoRa network | RF or Bluetooth |
| Resistance | | |
| Pod Optical Label | Included for easy wearer assignment and | Not included, no wearer |
| | identification | identification on pod |
| App | Intuitive web and mobile UX with Facility and | Mobile only, busy UX, |
| | Home level security | and/or home-use only |

In summary, the system and method of the present disclosure simplifies hardware setup, expands wearer detection range, reduces the need for frequent battery replacement or recharging, reduces the number of gateways needed to purchase and maintain, minimizes interference risks, and allows for easy pod assignment and identification compared to products currently on the market. The long-range wireless performance and cellular connectivity avoid reliance on facility infrastructure and prevent common issues seen in other monitoring devices.

### Example 1—Assisted Living Facility

The detection system can be easily set up at an assisted living facility to monitor a high volume of residents. First, the gateway is plugged in in a central location to maximize wireless coverage. No manual configuration or setup of the gateways is required. Next, the QR code on a pod is scanned to assign it to the appropriate resident in the related software application and is connected to an incontinence product on the resident. As the gateway automatically receives transmissions from the pod regarding wetness detections, notifications are seamlessly sent to a caregivers' device so that the caregiver can efficiently attend to resident's needs.

With the long-range LoRa wireless protocol covering the entire facility, caregivers can monitor residents remotely without having to manually check each incontinence product. The detection system operates reliably with cellular data and without relying on the facility's IT infrastructure or Wi-Fi. The caregivers benefit from an at-a-glance dashboard listing a current status for residents including those residents in need of attention, thereby allowing caregivers to quickly and efficiently identify and address residents needing immediate attention. By streamlining wetness detections and notifications, the detection system enables more attentive and proactive care while reducing disruptive checks on residents. The reliable LoRa wireless performance also eliminates gaps in monitoring as patients are transported around the facility. Standard of care improves, and caregivers maximize efficiency.

### Example 2—Home Health Care

For home health care scenarios, the system is equally applicable. A caregiver first plugs in the gateway in a central area of a client's home. The cellular connectivity means there is no need to rely on the home's internet connection. Next, the caregiver attaches a pod to the client's incontinence product and scans the QR code using the monitoring device or another device. Communication between the pod and gateway ensures notifications are successfully directed to the caregiver in real time.

As with use in a facility setting, the long-range wireless protocol enables remote monitoring from anywhere in the home without maintenance of batteries or charging. Notifications may pop up on the caregiver's device such that the caregiver can proactively attend to the client quickly, thereby preventing suboptimal health issues that result from wet incontinence products.

In home health scenarios, the system improves caregiver responsiveness and quality of care while allowing more dignity and independence for clients. It eliminates reliance on manual checks which may be insensitive and disruptive.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A detection system, comprising:
a pod including
a coupling for removably connecting the pod to an incontinence product,
a sensor configured to detect a wetness in the incontinence product, and
a pod transmitter configured to transmit a first signal, the first signal configured as a sub-gigahertz radiofrequency signal, the first signal including a first member selected from a group consisting of an indication of wetness in the incontinence product detected by the sensor, an identity of the pod, and combinations thereof;
a gateway including
a gateway receiver configured to receive the first signal from the pod, and
a gateway transmitter in communication with a network and configured to transmit a second signal upon receipt of the first signal, the second signal including a second member selected from a group consisting of the indication of wetness in the incontinence product detected by the sensor, the identity of the pod, the identity of the gateway and combinations thereof;
a server including
a server receiver configured to be in communication with the network and configured to receive the second signal from the gateway, and
a server transmitter configured to transmit a third signal using the network upon receipt of the second signal, the third signal including a third member selected from a group consisting of the indication of wetness in the incontinence product detected by the sensor, the identity of the pod, and combinations thereof; and
a device including
a device receiver configured to be in communication with the network and configured to receive the third signal from the server, and
a notification means configured to provide a notification based upon the third signal, the notification including a fourth member selected from a group consisting of the indication of wetness in the incontinence product detected by the sensor, the identity of the pod, and combinations thereof.

2. The detection system of claim 1, wherein the first signal is transmitted and received using a custom electronic data interchange protocol.

3. The detection system of claim 2, wherein the second signal is transmitted and received using the custom electronic data interchange protocol.

4. The detection system of claim 1, wherein the gateway is in cellular communication with the network.

5. The detection system of claim 1, wherein the server is in at least one of cellular Wi-Fi, and wired communication with the network.

6. The detection system of claim 1, wherein the device is in at least one of cellular and Wi-Fi communication with the network.

7. The detection system of claim 1, wherein the pod includes firmware designed to conserve battery consumption.

8. The detection system of claim 7, wherein the firmware enables a sleep mode during which the pod consumes no battery.

9. The detection system of claim 1, wherein the pod is configured to automatically connect to the gateway upon initiation of transmitting the first signal.

10. The detection system of claim 1, wherein the gateway and the pod each include a unique serial number for identification, auto-configuration and auto-registration.

11. The detection system of claim 1, wherein the gateway and the pod are updated remotely using the server.

12. The detection system of claim 1, wherein the sub-gigahertz radiofrequency signal is transmitted at 915 MHz.

13. The detection system of claim 1, wherein the sub-gigahertz radiofrequency signal is transmitted at 433 MHz.

14. The detection system of claim 1, wherein the notification means includes a software application installable on a device.

15. The detection system of claim 1, wherein the pod may include an optical label which may display a quick response code (QR).

16. The detection system of claim 1, wherein the server stores initialization data for automatically configuring the gateway and the pod.

17. The detection system of claim 1, wherein at least one of the first member and the second member is transmitted using a custom electronic data interchange protocol.

18. The detection system of claim 17, wherein the custom electronic data interchange protocol includes a unique key recognized only by the detection system.

19. A method of using a detection system, the method comprising steps of:
providing a pod having a coupling for removably connecting the pod to an incontinence product, a sensor configured to detect a wetness in the incontinence product, an optical label unique to the pod and assignable to a wearer, and a pod transmitter configured to transmit a first signal, the first signal configured as a sub-gigahertz radiofrequency signal, the first signal including a first member selected from a group consisting of an indication of wetness in the incontinence product detected by the sensor, an identity of the pod, and combinations thereof;
providing a gateway including a gateway receiver configured to receive the first signal from the pod and a gateway transmitter in communication with a network and configured to transmit a second signal upon receipt of the first signal, the second signal including a second member selected from a group consisting of the indication of wetness in the incontinence product detected by the sensor, the identity of the pod, identity of the gateway and combinations thereof;
providing a server including a server receiver configured to be in communication with the network and configured to receive the second signal from the gateway and a server transmitter configured to transmit a third signal using the network upon receipt of the second signal, the third signal including a third member selected from a group consisting of the indication of wetness in the incontinence product detected by the sensor, the identity of the pod, identity of the gateway and combinations thereof;
providing a device including a device receiver configured to be in communication with the network and configured to receive the third signal from the server and a notification means configured to provide a notification based upon the third signal, the notification including a fourth member selected from a group consisting of the indication of wetness in the incontinence product detected by the sensor, the identity of the pod, and combinations thereof;
installing the gateway at a desired location;
initiating communication between the gateway transmitter and the network by at least one of connecting the gateway to a power source and engaging a start function on the gateway;
auto-configuring the gateway using a first set of data from the server;
initiating the pod transmitter and the first signal by engaging a start function on the pod;
auto-configuring the pod using a second set of data from the gateway;
assigning the identity of the pod to the wearer using a unique QR code displayed on the pod;
coupling the pod to the incontinence product;
detecting the wetness in the incontinence product using the sensor;
sending the first signal from the pod to the gateway;
sending the second signal from the gateway to the server;
sending the third signal from the server to the device; and
monitoring the wetness in the incontinence product using the notification means.

20. The method of claim 19, wherein the first signal and the second signal are transmitted and received using a custom electronic data interchange protocol.
